(19) United States
(12) Patent Application Publication
(10) Pub. No.: US 2026/0365576 A1
(43) Pub. Date: Nov. 19, 2026

(54) GRINDING AID TABLET HAVING AN INTEGRATED STANDARD

(71) Applicants: thyssenkrupp Polysius GmbH, Beckum (DE); thyssenkrupp AG, Essen (DE)

(72) Inventors: Michael ENDERS, Muenster (DE); Jasmin HOLZER, Ennigerloh (DE)

(73) Assignees: thyssenkrupp Polysius GmbH, Beckum (DE); thyssenkrupp AG, Essen (DE)

(21) Appl. No.: 19/133,549

(22) PCT Filed: Nov. 23, 2023
(86) PCT No.: PCT/EP2023/082821

§ 371 Date: May 28, 2025

(30) Foreign Application Priority Data
Dec. 2, 2022 (DE) 10 2022 132 071.5
Dec. 2, 2022 (LU) LU103044

(51) Int. Cl. C04B 24/38; C04B 14/04; C04B 14/30; C04B 18/02; C04B 24/26; C04B 24/34; C04B 103/52

(52) U.S. Cl. CPC C04B 24/383; C04B 14/043; C04B 14/305; C04B 18/022; C04B 24/2641; C04B 24/34; C04B 2103/52

(57) ABSTRACT

A grinding aid has the following composition: (a) 15% to 40% of cellulose or cellulose derivative, (b) 40% to 60% of natural resin, (c) 0% to 5% of auxiliaries and coating substances, and (d) 15% to 25% of crystalline inorganic material as X-ray standard, where the sum of the components (a), (b), (c), and (d) comes to 100%, all values being in % by weight.

The invention relates to a grinding aid tablet with an integrated crystalline substance as standard for the quantitative determination by X-ray diffraction of amorphous constituents, for example for the production of clinker and cement.

Samples in the cement industry, the aluminum industry or in steel production, for example, are nowadays often quantitatively analyzed by X-ray diffractometric methods, which are sometimes automated for process control. For quantitative evaluation, a Rietveld adjustment is performed, which permits quantitative determination of the phases in relation to one another. In particular, the use here of area detectors for process control permits sensibly short measurement times for quantitative analysis with accuracy down to 0.1% for the individual phase fractions. Difficulties arise here in the quantification of amorphous compounds, which are accordingly routinely adjusted via what is known as a pseudostructure.

Grinding aids, more particularly grinding aid tablets, have three primary functions. Firstly, the grinding aids aid the comminuting effect, which allows better homogenization of the sample to be achieved during grinding. Secondly, the grinding aids are intended to make automated cleaning of the grinding units easier. In purely automated sample production in particular, this allows contamination between successive samples to be minimized by achieving complete discharge. These constituents of the grinding aid tablet then also provide a third effect of stabilizing the pressed sample pellet that is produced, to ensure it can be easily investigated and handled. Grinding aids are therefore usually added in an order of about 10% by weight.

The use of grinding aids in the form of grinding aid tablets permits good, in particular automated, processing, very simple and reliable dosing, and avoids problems with contamination.

DE 10 2013 106 998 A1 discloses a process and a device for producing a pellet.

DE 10 2020 201 877 B3 discloses a composition of a grinding aid tablet.

The materials of the grinding aids are usually amorphous and thus contribute to an amorphous substrate generally already present. This makes it difficult to quantify amorphous constituents in a sample.

In addition to the three requirements already mentioned that must be met by the grinding aid tablet, a further requirement is of course that the grinding aid tablet must itself be stable in order for reliable and, in particular, automated processing to be possible.

The problem addressed by the invention is that of being able to quantitatively determine as far as possible all phases of the sample.

This problem is solved by the grinding aid having the features specified in claim 1 and by the method having the features specified in claim 8. Advantageous developments will be apparent from the dependent claims and from the description that follows.

The grinding aid of the invention serves to make it possible to grind a sample more readily, particularly in an automated manner, and to produce a stable X-ray analysis pellet for subsequent investigation by X-ray diffraction. The function of the grinding aid here is to aid grinding and thus good homogenization of the sample, to aid the complete removal of the sample from the equipment, and then to impart stability to the pressed X-ray analysis pellet. In addition, a further problem addressed by the invention is that of providing an internal X-ray standard in order that a quantitative analysis is reliably possible.

According to the invention, the grinding aid has the following composition:
A 15% to 35% of cellulose or cellulose derivative,
B 40% to 60% of natural resin,
C 0% to 5% of auxiliaries and coating substances,
D 15% to 25% of crystalline inorganic material as X-ray standard.

The sum of components A, B, C, and D comes to 100%. All values here and below are in % by weight.

Cellulose derivatives (A) are chemical derivatives of cellulose and are commonly used as binders, for example in pharmaceutical and cosmetic products. Cellulose derivatives are prepared for example by methylation (methylcellulose), ethylation, hydroxypropylation, sulfonation, nitration (cellulose nitrate), acetylation (cellulose acetate), oxidation, xanthogenation, crosslinking, or copolymerization through grafting. There are numerous further modifications.

Natural resin (B) helps stabilize a sample pellet through its stickiness in particular.

Auxiliaries and coating substances (C) are used for example to protect the surface of the grinding aid tablet, to improve pouring properties, and the like. Such coatings and coating processes are familiar to those skilled in the art. Because of their small proportion, these substances have little, optimally no, effect on the grinding process and in the subsequent investigation of the ground material.

The composition thus differs in respect of the additional constituent D from conventional grinding aids, such as those known from DE 10 2020 201 877B3, for example.

However, the addition of crystalline inorganic material necessitates a larger amount of grinding aid overall. The altered composition in turn also causes in particular an altered ratio between component A and component B. If the higher proportion of cellulose disclosed in the prior art were to be used, the X-ray analysis pellet subsequently produced would be less stable, with a tendency to break by lamination. It is therefore not possible to simply add a crystalline inorganic material without adjusting the further formulation.

Therefore, it is also advantageous when the crystalline inorganic material has already been added to the grinding aid. Firstly, during subsequent use, the second dosing step for separate addition of an internal standard in the form of a crystalline inorganic material becomes superfluous. Secondly, the ratio between the other components of the grinding aid and the crystalline inorganic material is also as a result constant and independent of, for example, dosing or weighing errors. The composition of the further components of the grinding aid can therefore be precisely adjusted in line with the preset ratio between the other components of the grinding aid and the crystalline inorganic material.

As well as X-ray diffraction, other investigations can also be carried out on the X-ray analysis pellet, either at the same time or spaced apart. For example, a composition may be investigated by X-ray fluorescence. An investigation by infrared spectroscopy may optionally also be performed. This is a further advantage of the X-ray analysis pellet: it is possible to perform a plurality of non-destructive investigations on the same X-ray analysis pellet.

In another embodiment of the invention, component A and component B are present in a ratio to one another of 25:75 to 35:65. This achieves optimum strength while minimizing the friability of the X-ray analysis pellet. At the same time, good self-cleaning of the mill is also achieved. This ratio differs significantly from a grinding aid without crystalline inorganic material, as disclosed for example in DE 10 2020 201 877B3. This ratio has proved to be optimal for the high proportion of crystalline inorganic material that is necessary.

In another embodiment of the invention, component D comprises one or more substances from the list comprising titanium dioxide, especially rutile, aluminum oxide, especially corundum, silicon dioxide, especially quartz, silicon carbide, silicon, hematite, and magnetite. The substance selected is preferably not also a constituent of the sample. Typical areas of application of automated X-ray diffraction for process control are nowadays found for example in the cement industry, in iron and steel production, especially in the field of slags, in aluminum production or in titanium dioxide production. Those skilled in the art will select a suitable crystalline inorganic material that in the diffractogram has the least overlap with the peaks of the crystalline material of the sample.

In another embodiment of the invention, the proportion of component A is between 28% and 32%, preferably 30%.

In another embodiment of the invention, the natural resin of component B comprises the following components:
a 3 to 20% of 2,4-bis(1-phenylethyl)phenol,
b 1 to 12% of dehydroabietic acid,
c 1 to 8% of 1,2-bis(4-methoxyphenyl)-1-propene,
d 1 to 7% of ((1R, 4aR, bR, 7R, 10aR)-1,4a,7-trimethyl-7-vinyl-1,2,3,4,4a,4b,6,7,8,9,10,10a-dodecahydrophenanthren-1-yl)-methanol,
e 1 to 7% of (1R, 4aR, bR, 7R, 10aR)-1,4a,7-trimethyl-7-vinyl-1,2,3,4,4a,4b,6,7,8,9,10,10a-dodecahydrophenanthrene-1-carbaldehyde.

The sum of all the components of the natural resin comes to 100%.

Complete analysis of all components is of course not possible and, for substances present in very low proportion in particular, significant fluctuations can occur. Moreover, the natural resin exhibits fluctuations from one region to the next and from one year to the next, consequently it is not possible to specify an exact composition. Fluctuations occur for example as a consequence of temporal, regional or climatic differences in plant growth.

The natural resin was firstly investigated by 1D-NMR and 2D-NMR. Secondly, a pyrolysis at 450 °C was carried out for 10 s and the products then via a HP-5 ms GC column with a temperature program of 45-240 °C at 4 K/min and then 240-300 °C at 40 K/min and a hold time of 15 min, with helium as carrier gas with a flow rate of 1 ml/min at a split of 50:1. The separated substances were identified by mass spectroscopy, with a scan from 10 to 600 g/mol carried out.

In another embodiment of the invention, the natural resin of component B additionally comprises the following components:
f 0.5 to 6% of longifolene,
g 0.5 to 6% of abietic acid,
h 0.5 to 5% of caryophyllene oxide.

In another embodiment of the invention, the natural resin of component B additionally comprises the following components:
i 2 to 25% of 9-ethyl-1,2,3,4,5,6,7,8-octahydroanthracene,
j 2 to 25% of N-(2-ethylphenyl)-phenylacetamide.

In another embodiment of the invention, the natural resin of component B comprises the following component:
b 3 to 8% of dehydroabietic acid.

In another embodiment of the invention, the natural resin of component B comprises the following components:
d 2 to 5.5% of ((1R, 4aR, bR, 7R, 10aR)-1,4a,7-trimethyl-7-vinyl-1,2,3,4,4a,4b,6,7,8,9,10,10a-dodecahydrophenanthren-1-yl)-methanol,
e 2 to 5.5% of (1R, 4aR, bR, 7R, 10aR)-1,4a,7-trimethyl-7-vinyl-1,2,3,4,4a,4b,6,7,8,9,10,10a-dodecahydrophenanthrene-1-carbaldehyde.

In another embodiment of the invention, the grinding aid has a coating of PMMA. This makes handling easier and at the same time reduces material losses during handling, for example due to abrasion or chipping.

In another embodiment of the invention, the grinding aid is a grinding aid tablet having a mass of 0.2 to 0.4 g. With customary sample sizes of around 10 g (usually 5 g to 20 g) found to be suitable in order to have exact dosing and in order at the same time for the number of grinding aid tablets needed, and accordingly the dosing outlay, not to be too high.

In another aspect, the invention relates to a method for the quantitative determination of the phase fractions of a sample by X-ray diffraction. The method comprises the following steps:
a) weighing the sample,
b) adding the grinding aid as claimed in any of the preceding claims,
c) grinding,
d) pressing an X-ray analysis pellet,
e) recording the X-ray diffractogram,
f) Rietveld adjustment of the X-ray diffractogram.

According to the invention, the method has the characteristic feature that
g) the quantitative proportion of the amorphous phase of the sample is determined via the ratio of the crystalline phase of the sample in relation to the crystalline inorganic material of the grinding aid.

The sample weight in step a) can be either exact or approximate. In the case of an exact sample weight, a specification of for example 10.00 g is specified and exactly this mass of sample is weighed out. Alternatively, in the case of an approximate sample weight, an amount of about 10 g, for example of between 8 g and 12 g can be specified, the exact mass, for example 11.11 g, then being measured when the sample is provided exactly. Both procedures have their advantages and disadvantages.

The dosing in step b) is particularly straightforward when grinding aids are present in the form of grinding aid tablets, for example in a mass of 250 mg per grinding aid tablet. If, for example, 10 g of sample is specified, then 4 grinding aid tablets totaling 1 g are added by way of example. The number four is relatively easy to count and dose, and the measurement error remains minimal as a result.

This is then followed by the grinding in step C) and the pressing of an x-ray analysis pellet in step d).

The X-ray analysis pellet produced in step d) is investigated by X-ray diffraction in step e), for example using a CuKa source and a 2D area detector. It is essential that the X-ray diffractogram recorded in step e) is of sufficient quality for the Rietveld adjustment that follows in step f).

The basis of the Rietveld method of adjustment is to specify the known phases and their known reflections (position and intensity ratio). The key parameters for the investigation are then varied, in particular the ratio of the phases to one another, but also, for example, the particle size (which can result in broadening of the reflections). The latter may be particularly suitable for calculating an amorphous or pseudoamorphous fraction, for example. A comparison is then made between the simulated diffractogram and the diffractogram recorded in step e), the optimum being determined by the least-squares method. This allows the ratio of the different crystalline phases to one another to be determined very well, with the result that quantitative investigation of the phases is possible. For example, in a clinker the ratio of alite (C₃S) to belite (C₂S) can be determined in this way.

Since the amorphous fraction is difficult to determine and is also superimposed by the amorphous constituents of the grinding aid, the amorphous fraction is quantitatively determined from the ratio of the proportion of the crystalline sample to the proportion of the crystalline inorganic material as X-ray standard. This will be explained by way of example as follows.

For example, 10 g of sample is processed with 1 g of grinding aid, where the grinding aid includes 20% of rutile, i.e. 0.2 g of rutile. Based on a purely hypothetical and simplified assumption that the sample comprises equal parts of alite and belite, a completely crystalline sample would have an alite: belite: rutile ratio of 25:25:1. If a ratio of 12.5:12.5:1 is obtained in the Rietveld analysis, then only half of the sample can be crystalline. In this very simple hypothetical case, this means that the quantitative proportion of the amorphous phase in the sample would be 50%.

What makes this so simple is that, through the use of a grinding aid with an internal standard, it is possible to have clear relationships in a simple manner, even taking into consideration the amorphous constituents of the grinding aid.

In another embodiment of the invention, dosing with 0.07 to 0.12 g of grinding aid per 1 g of sample is carried out in step b). There is a tendency toward higher dosing, on account of the additional crystalline inorganic material, but this should not be too high, in order to ensure that the X-ray analysis pellet still has good properties and also that superimposition is not too great in the X-ray diffractogram of the sample.

In another embodiment of the invention, rutile is used as crystalline inorganic material for a cement sample or cement clinker sample.

In another embodiment of the invention, corundum, silicon or silicon carbide is used as crystalline inorganic material for a titanium dioxide sample.

1-11. (canceled)

12. A grinding aid having the following composition:
(A) 15% to 35% of cellulose or cellulose derivative;
(B) 40% to 60% of natural resin;
(C) 0% to 5% of auxiliaries and coating substances; and
(D) 15% to 25% of crystalline inorganic material as X-ray standard;
where a sum of components (A), (B), (C), and (D) comes to 100%, all values being in % by weight.

13. The grinding aid as claimed in claim 12, wherein component (A) and component (B) are present in a ratio to one another of 25:75 to 35:65.

14. The grinding aid as claimed in claim 12, wherein component (D) comprises one or more substances from the list consisting of: titanium dioxide, aluminum oxide, silicon dioxide, silicon carbide, silicon, hematite, and magnetite.

15. The grinding aid as claimed in claim 12, wherein component (D) comprises one or more substances from the list consisting of: rutile, corundum, quartz, silicon carbide, silicon, hematite, and magnetite.

16. The grinding aid as claimed in claim 12, wherein the proportion of component (A) is between 28% and 32%.

17. The grinding aid as claimed in claim 12, wherein the natural resin of component (B) contains the following components:
(a) 3% to 20% of 2,4-bis(1-phenylethyl)phenol;
(b) 1% to 12% of dehydroabietic acid;
(c) 1% to 8% of 1,2-bis(4-methoxyphenyl)-1-propene;
(d) 1% to 7% of ((1R, 4aR, bR, 7R, 10aR)-1,4a,7-trimethyl-7-vinyl-1,2,3,4,4a, 4b,6,7,8,9,10,10a-dodecahydrophenanthren-1-yl)-methanol; and
(e) 1% to 7% of (1R, 4aR, bR, 7R, 10aR)-1,4a,7-trimethyl-7-vinyl-1,2,3,4,4a,4b,6,7,8,9,10,10a-dodecahydrophenanthren-1-carbaldehyde;
where a sum of all the components of the natural resin comes to 100%.

18. The grinding aid as claimed in claim 12, wherein the grinding aid has a coating of PMMA.

19. The grinding aid as claimed in claim 12, wherein the grinding aid is a grinding aid tablet having a mass of 0.2 to 0.4 g.

20. A method for quantitative determination of phase fractions of a sample by X-ray diffraction, comprising:
(a) weighing the sample;
(b) adding the grinding aid as claimed in claim 12;
(c) grinding;
(d) pressing an X-ray analysis pellet;
(e) recording an X-ray diffractogram; and
(f) Rietveld adjustment of the X-ray diffractogram;
wherein a quantitative proportion of an amorphous phase of the sample is determined via a ratio of a crystalline phase of the sample to a crystalline inorganic material of the grinding aid.

21. The method as claimed in claim 20, wherein dosing with 0.07 to 0.12 g of grinding aid per 1 g of sample is carried out in step (b).

22. The method as claimed in claim 20, wherein rutile is used as crystalline inorganic material for a cement sample or cement clinker sample.

23. The method as claimed in claim 20, wherein corundum, silicon or silicon carbide is used as crystalline inorganic material for a titanium dioxide sample.
